(19) United States
(12) Patent Application Publication
(10) Pub. No.: US 2026/0232882 A1
(43) Pub. Date: Aug. 13, 2026

(54) SYSTEM USING BALANCING CHAMBERS

(71) Applicant: DIALITY INC., Irvine, CA (US)

(72) Inventor: Colin Weaver, Irvine, CA (US)

(21) Appl. No.: 19/436,245

(22) Filed: Dec. 30, 2025

Related U.S. Application Data
(60) Provisional application No. 63/741,763, filed on Jan. 3, 2025.

(51) Int. Cl. A61M 1/16; A61M 1/28

(52) U.S. Cl. CPC A61M 1/1694; A61M 1/1601; A61M 1/1666; A61M 1/1672; A61M 1/281; A61M 1/287; A61M 2205/3334

(57) ABSTRACT

Disclosed are example embodiments of a dialysis system that enables real-time preparation and delivery of peritoneal dialysis solution using a balancing chamber architecture. The system includes a water purification module configured to convert tap water into purified water meeting dialysis quality standards, and a mixing subsystem configured to combine the purified water with an osmotic agent, electrolytes, and a buffer to produce peritoneal dialysis solution on demand. A balancing chamber having a fresh side and a spent side is used to precisely meter delivery of the solution to a patient, with pressure applied to the spent side through a recirculation pathway. The system further includes fluid pathways for delivering the prepared solution to the patient and for removing fluid from the patient, under control of a controller that manages fluid flow and dosing accuracy. By integrating real-time solution preparation with balancing chamber-based fluid control, the system improves flexibility, reduces reliance on pre-mixed solutions, and enhances treatment efficiency.

### CROSS-REFERENCE TO RELATED APPLICATION

This application claims priority to U.S. Provisional Patent Application No. 63/741,763, filed on Jan. 3, 2025, which is incorporated herein by reference in its entirety for all purposes.

### TECHNICAL FIELD

The present disclosure relates generally to dialysis treatment systems and, more specifically, to systems and methods for utilizing a balancing chamber to enable real-time preparation of peritoneal dialysis solutions and advanced fluid management in dialysis machines.

### BACKGROUND

Dialysis is a life-sustaining procedure that removes waste products, excess fluids, and toxins from the blood of patients with renal failure. Hemodialysis and peritoneal dialysis are two primary forms of this treatment, each requiring precise fluid management and high-quality dialysis solutions. Traditional systems rely on pre-mixed dialysis solutions, which necessitate extensive storage, transportation, and strict quality control to ensure patient safety. Additionally, existing dialysis machines are primarily designed for specific modalities, limiting their adaptability and efficiency in addressing diverse treatment needs.

Accordingly, there is a need for improved methods and systems for real-time preparation of dialysis solutions directly from tap water, enabling advanced fluid management through the integration of balancing chambers and adaptable architectures for both peritoneal and hemodialysis applications.

### SUMMARY

In example embodiments, the present disclosure relates to systems and methods for improving dialysis treatment by enabling flexible fluid management architectures and on-demand preparation of dialysis solutions. The disclosed approaches may support real-time solution preparation, controlled fluid delivery and removal, and adaptability across multiple dialysis modalities, including peritoneal dialysis and hemodialysis. In some implementations, existing dialysis system architectures may be leveraged or adapted to support additional therapies or treatment workflows. The disclosed systems and methods are intended to reduce reliance on pre-prepared solutions, improve operational efficiency, and enhance treatment flexibility, while maintaining appropriate safety and quality considerations.

Disclosed are example embodiments of a dialysis system that enables real-time preparation and delivery of peritoneal dialysis solution using a balancing chamber architecture. The system includes a water purification module configured to convert tap water into purified water meeting dialysis quality standards, and a mixing subsystem configured to combine the purified water with an osmotic agent, electrolytes, and a buffer to produce peritoneal dialysis solution on demand. A balancing chamber having a fresh side and a spent side is used to precisely meter delivery of the solution to a patient, with pressure applied to the spent side through a recirculation pathway. The system further includes fluid pathways for delivering the prepared solution to the patient and for removing fluid from the patient, under control of a controller that manages fluid flow and dosing accuracy. By integrating real-time solution preparation with balancing chamber-based fluid control, the system improves flexibility, reduces reliance on pre-mixed solutions, and enhances treatment efficiency.

Disclosed are example embodiments of a method for performing peritoneal dialysis using a balancing chamber system and real-time solution preparation. The method includes purifying tap water to produce water meeting dialysis quality standards and mixing the purified water with an osmotic agent, electrolytes, and a buffer to generate peritoneal dialysis solution in real time. The method further includes pressurizing a spent side of a balancing chamber using a recirculation pathway to control movement of a barrier within the chamber, delivering solution from a fresh side of the balancing chamber to a patient, and removing fluid from the patient using a dedicated pump. The method supports precise fluid management while enabling on-demand solution preparation, thereby reducing storage and logistical requirements associated with pre-mixed dialysis fluids.

### BRIEF DESCRIPTION OF THE DRAWINGS

The foregoing summary, as well as the following detailed description, is better understood when read in conjunction with the accompanying drawings. The accompanying drawings, which are incorporated herein and form part of the specification, illustrate a plurality of embodiments and, together with the description, further serve to explain the principles involved and to enable a person skilled in the relevant art(s) to make and use the disclosed technologies.

FIG. 1 is a schematic diagram illustrating the standard fluid path used in a hemodialysis device, as adapted for peritoneal dialysis.

FIG. 2 is a schematic diagram illustrating the fresh peritoneal dialysis (PD) solution delivery pathway to the patient using a balancing chamber mechanism.

FIG. 3 is a schematic diagram illustrating the recirculation-based pressurization of the spent side of the balancing chamber to ensure precise fluid management.

FIG. 4 is a schematic diagram illustrating the fluid removal pathway from the patient, incorporating a dedicated pump for precise fluid extraction.

FIG. 5 is a schematic diagram illustrating the adaptation of the hemodialysis architecture to support a four-stream peritoneal dialysis chemistry, including the bifurcation of the acid line and the integration of additional valves, in accordance with the systems and methods described herein.

FIG. 6 illustrates an example flow diagram of a method for performing peritoneal dialysis.

The figures and the following description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

### DETAILED DESCRIPTION

The detailed description set forth below in connection with the appended drawings is intended as a description of configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well-known structures and components are shown in block diagram form in order to avoid obscuring such concepts.

The present disclosure relates to systems and methods for pushing dialysate across a dialyzer using a balancing chamber system. In certain embodiments, the systems and methods allow a dialysis machine to perform alternative dialysis therapies by directing dialysate toward the blood-side fluid path of a dialysis machine. In further embodiments, the systems and methods enable the preparation of peritoneal dialysis (PD) solution in real time using tap water, while maintaining high water quality standards. The apparatuses and processes described herein may be implemented using the architecture of existing hemodialysis (HD) machines with modifications primarily directed to fluid pathway control and software operation.

In conventional systems, dialysis machines maintain balanced flow on the dialysate side of a dialyzer in order to achieve accurate ultrafiltration performance. This balance is commonly achieved through the use of a balancing chamber that equalizes dialysate inflow and dialysate outflow. However, a number of dialysis procedures and therapies require transfer of fluid from the dialysate-side of the fluid circuit to the blood-side. Such procedures may include priming of blood tubing sets, administering fluid boluses to a patient during therapy, rinse-back of blood at the end of treatment, convective therapies including hemofiltration and hemodiafiltration, and peritoneal dialysis applications. Existing architectures do not readily permit intentional shifting of fluid from the dialysate-side to the blood-side, particularly when the balancing mechanism is configured to force equivalence between dialysate inflow and outflow streams. The systems and methods described herein provide configurations for bypassing or re-purposing the balanced system to create directed flow from the dialysate-side of the machine through the dialyzer and toward fluid pathways used for blood or peritoneal delivery.

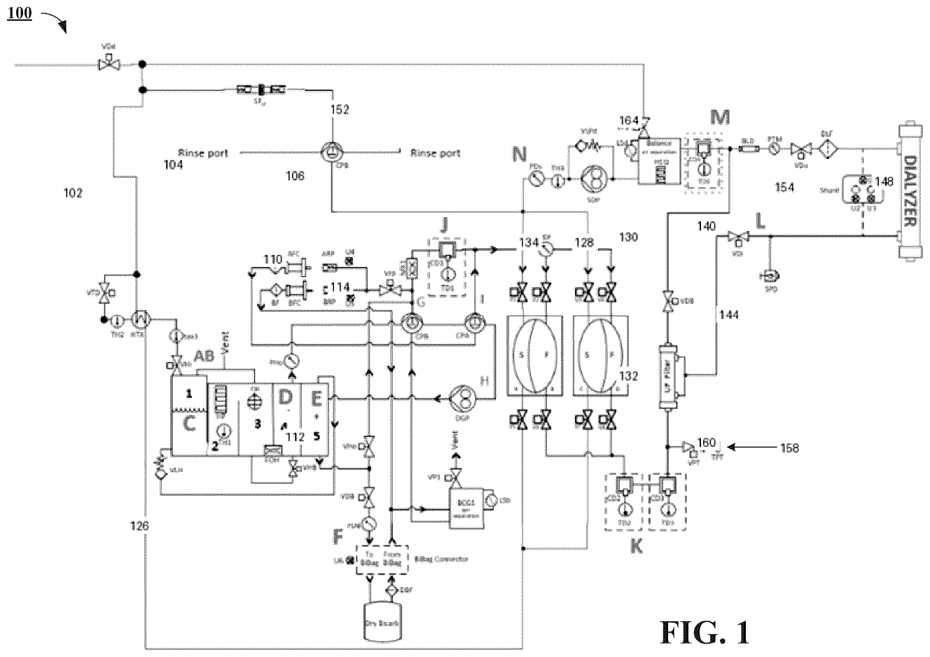

In some embodiments, a peritoneal dialysis device and method are provided that utilize a balancing chamber system to enable precise fluid handling. The device may further include a capability to mix peritoneal dialysis solution in real time directly from tap water. FIG. 1 illustrates an example hydraulic flow diagram representative of a standard HD fluid path architecture that may serve as the foundation for the systems described in this disclosure. Using this architecture as a base, the peritoneal dialysis functionality can be implemented with minimal hardware changes while enabling substantial expansion of therapy options.

FIG. 1 illustrates an example hemodialysis fluid path architecture 100 adapted to support peritoneal dialysis and dialysate-to-blood-side transfer using a balancing chamber system. As shown in FIG. 1, tap water is received at a water inlet 102 and directed into a water purification and heater assembly 104. The water purification and heater assembly 104 may include filters, temperature-control elements, and safety interlocks and is configured to produce purified water meeting dialysis water quality standards. Downstream of the assembly 104, the circuit includes an air-removal and vent region 106 that may incorporate structures to remove entrained gas prior to chemical proportioning. In a central portion of FIG. 1, a mixing and proportioning subsystem 110 receives purified water from the assembly 104 together with one or more concentrates, including an osmotic agent, electrolytes, and a buffer. In some embodiments, the subsystem 110 is supplied with dry bicarbonate or an alternative concentrate via a bicarbonate cartridge system 112. A proportioning assembly 114 within the subsystem 110 may include concentrate pumps, metering devices, and associated valves that control the ratio of purified water to concentrates to generate dialysate or peritoneal dialysis solution of a prescribed chemistry. Operation of the proportioning assembly 114 and associated components is coordinated by a controller 126 that monitors solution preparation and enforces alarm and shutoff conditions.

Downstream of the mixing and proportioning subsystem 110, the fluid pathway enters a balancing chamber assembly 128. The balancing chamber assembly 128 may include one or more balancing chambers, each having a fresh-fluid compartment 130 and a spent-fluid compartment 132 separated by a movable barrier such as a diaphragm, membrane, or plate. Valve sets 134 disposed upstream and downstream of the balancing chamber assembly 128 direct fresh solution and spent effluent into corresponding compartments 130, 132 on alternating cycles so that substantially equal volumes are exchanged. During conventional hemodialysis operation, this arrangement maintains balance between dialysate inflow and outflow while supporting accurate ultrafiltration performance.

A fresh-solution outlet line 140 extends from the fresh-fluid compartments 130 of the balancing chamber assembly 128 toward the right-hand side of FIG. 1. Along this path, the solution may pass through an ultrafiltration module 144, which may be implemented as a dual ultrafiltration system to remove bacteria, endotoxin, and particulates before the solution is presented to a patient. In some embodiments, the outlet line 140 is routed through or around a dialyzer 148. The dialyzer 148 may function in its conventional role for hemodialysis or, in peritoneal dialysis and other therapies, may provide an additional filtration barrier when solution is intentionally driven across the dialyzer membrane. One or more rinse ports 152 are located along upper portions of the circuit and permit fresh water or chemical disinfectant to be introduced for priming, rinsing, or disinfection without disassembling the internal tubing.

A spent-solution pathway 154 collects dialysate or peritoneal effluent exiting the dialyzer 148 and returns it toward the spent-fluid compartments 132 of the balancing chamber assembly 128. After passing through the balancing chamber assembly 128, a drain line 158 routes spent fluid toward a building drain or collection container. A flow-diverter 160 is positioned along the drain line 158 and may be actuated to direct fluid selectively toward a downstream disinfection and test region or directly toward the external drain connection. In this manner, the same drain pathway 158 can support normal dialysis operation, chemical disinfection, and solution sampling while maintaining a compact overall layout.

In addition to the main dialysate loop, the architecture 100 includes a recirculation pathway that overlays the existing fluid path. A recirculation pump 164 is arranged so that, under control of the controller 126, dialysate or other fluid can be circulated around a loop that communicates hydraulically with the spent-fluid compartments 132 of the balancing chamber assembly 128. When the recirculation pump 164 is operated with appropriate valve states, pressure generated in this loop acts on the spent-fluid compartments 132 and displaces the movable barriers within the balancing chambers. This displacement meters a corresponding volume of fresh solution from the fresh-fluid compartments 130 toward the outlet line 140 and, ultimately, toward a patient connection. During rinsing and disinfection, the same recirculation pathway may be used to distribute rinsing fluid through the internal circuit while the patient is disconnected.

The arrangement of FIG. 1 allows peritoneal dialysis functionality to be implemented primarily through reconfiguration of fluid pathways and control logic rather than wholesale redesign of the hemodialysis machine. The water purification assembly 104 and mixing subsystem 110 generate peritoneal dialysis solution in real time from tap water and concentrates. The balancing chamber assembly 128, which traditionally serves to enforce equal inflow and outflow volumes, is additionally used as a precision dosing structure when its spent-fluid compartments 132 are pressurized via the recirculation pump 164. The dialyzer 148 may be included or bypassed depending on the selected therapy mode, and the drain line 158 together with the diverter 160 provides a controlled route for discharge, disinfection, and sampling of spent fluid. The controller 126 coordinates these components to support hemodialysis, hemodiafiltration, hemofiltration, dialysate-to-blood-side transfers, and peritoneal dialysis within the same base architecture.

With continued reference to FIG. 1, the overall architecture 100 is laid out so that the water inlet 102 and water purification and heater assembly 104 appear at an upper-left portion of the drawing, followed by the air-removal region 106 and the mixing and proportioning subsystem 110 in a central-left block. The bicarbonate cartridge system 112 is located beneath the subsystem 110, and the balancing chamber assembly 128 is depicted near the center-right of the diagram as a pair of chambers with associated valve sets 134. The ultrafiltration module 144 is shown to the right of the balancing chamber assembly 128, and the dialyzer 148 is located at the far right. Rinse ports 152 are positioned along the upper horizontal run of the circuit. The drain line 158 and flow-diverter 160 appear in a lower-right region of the architecture, and the recirculation pump 164 is positioned along an upper central portion of the diagram, coupled by internal plumbing to the spent-fluid compartments 132 of the balancing chamber assembly 128.

In certain embodiments, purified water meeting Association for the Advancement of Medical Instrumentation (AAMI) quality criteria is first produced from tap water. The water may be processed using one or more stages including heating, degassing, and filtration or other purification technologies. The purified water may then be combined with an osmotic agent, electrolytes, and a buffer to produce PD solutions in real time. The solution may thereafter pass through a dual ultrafiltration (UF) system configured to remove bacteria, endotoxins, and other contaminants to render the solution ultra-pure and suitable for administration to a patient. The mixing and purification processes may be implemented in a continuous or batch mode under software control.

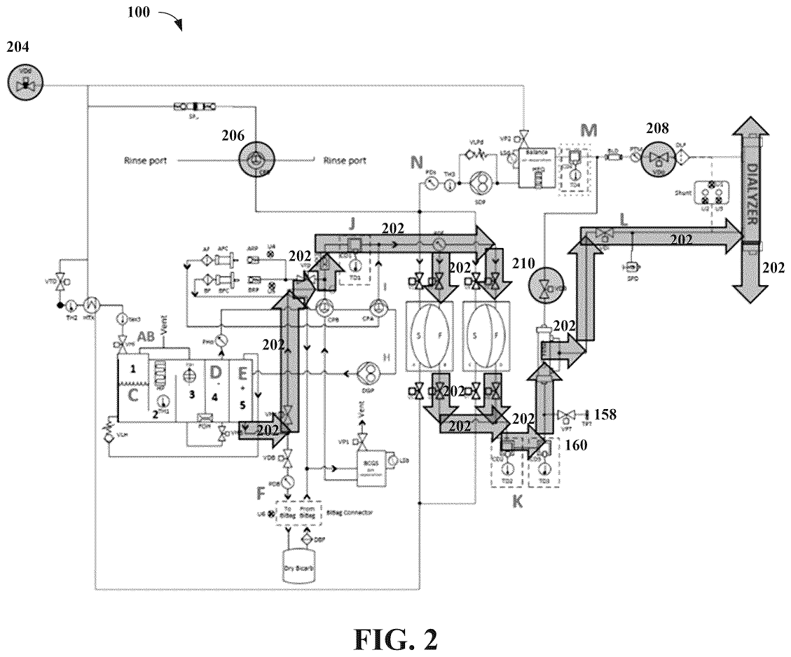

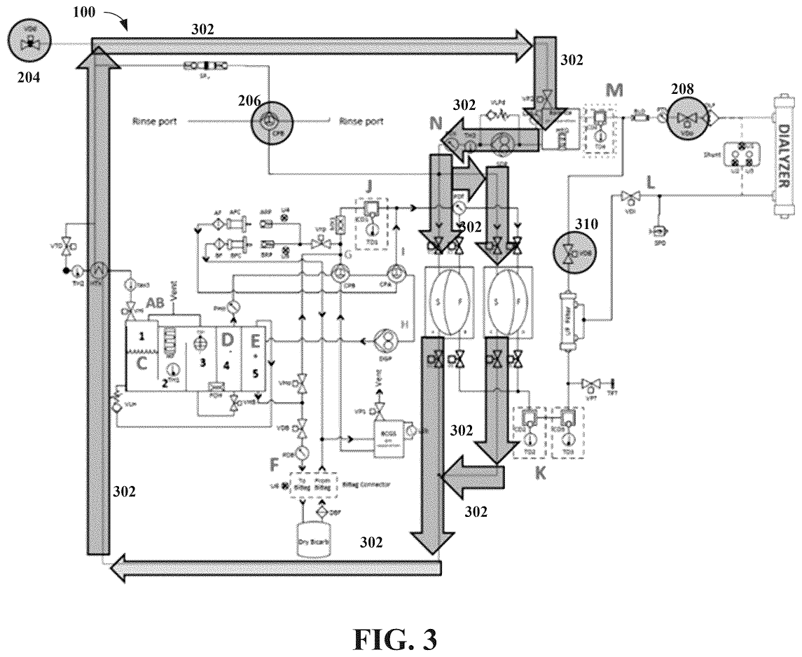

In some embodiments, the balancing chamber is configured to deliver PD solution to the patient. FIG. 2 illustrates an example of a fresh fluid delivery path from the balancing chamber to the patient. The balancing chamber operates by maintaining pressure or volume equivalence between a fresh-fluid side and a spent-fluid side. In certain embodiments, the spent side of the balancing chamber is pressurized by recirculating fluid along a recirculation pathway such as that shown in FIG. 3. By recirculating fluid on the spent side, the balancing chamber may be functionally converted into a dosing pump for controlled delivery of fresh PD solution.

FIG. 2 illustrates an example fresh fluid delivery pathway from the balancing chamber assembly 128 toward a patient connection. A delivery pathway 202 extends from a region downstream of the balancing chamber assembly 128, passes through valve sets adjacent the fresh-fluid compartments 130, and continues toward a dialyzer 148 and a patient connection set. Delivery pathway 202 may be implemented using flexible tubing, rigid conduits, or cassette-based channels and is arranged as a generally looped structure that overlays portions of the existing hemodialysis fluid path. During peritoneal dialysis operation, fresh peritoneal dialysis solution exiting the fresh-fluid compartments 130 is routed along pathway 202 and is displaced toward the patient in response to pressure applied on the spent-fluid compartments 132, as described above.

Delivery pathway 202 includes multiple vertical and horizontal segments that route along the front of the balancing chamber assembly 128 and then toward the right-hand side of the architecture. In the region labeled J in FIG. 2, pathway 202 branches from the balancing chamber outlet and is directed upward and across the top of the balancing chambers. Additional segments of pathway 202 extend downward and then upward again in the region labeled K, creating a stepped configuration that accommodates valves and monitoring structures while preserving hydraulic continuity. At the right-hand side of FIG. 2, pathway 202 extends horizontally through a region labeled L toward the dialyzer 148 and then downward toward a patient connection set, where multiple connection points are provided for fluid lines leading to a peritoneal catheter or to other extracorporeal circuits.

Valve 204 is positioned along an upper boundary line that defines an interface between the fresh-solution delivery pathway 202 and an upstream portion of the dialysate circuit. Valve 204 may, for example, be actuated to connect or isolate this upper boundary line from an external supply or drain during rinsing, disinfection, or system priming. In some embodiments, valve 204 remains closed during peritoneal dialysis operation so that delivery pathway 202 functions as a dedicated route from the balancing chamber assembly 128 toward the dialyzer 148 and patient, while valve 204 is opened during cleaning modes to permit flushing of the pathway 202 and associated structures.

Valve 206 is located along a branch leading from a rinse port 152 to the upper boundary line controlled by valve 204. Valve 206 may be opened to admit rinse solution or disinfectant from the rinse port into the upper boundary line and, through appropriate valve states, into delivery pathway 202. In this configuration, the same physical pathway used to deliver peritoneal dialysis solution to the patient can be flushed using externally supplied rinse fluid without requiring separate tubing sets. Valve 206 may be closed during normal therapy to prevent unintended ingress of external fluid into the delivery pathway 202.

Valve 210 is positioned adjacent the balancing chamber assembly 128 along a junction where flow from the fresh-fluid compartments 130 is directed into delivery pathway 202. Valve 210 may, for example, control whether fresh solution exiting the balancing chambers is routed into pathway 202 for peritoneal dialysis delivery, directed back into a conventional hemodialysis dialysate loop, or blocked entirely. In a peritoneal dialysis mode, valve 210 is opened to allow fresh solution to enter pathway 202, while in a hemodialysis mode valve 210 may be set so that the balancing chambers operate in their traditional role of balancing dialysate inflow and outflow without feeding the peritoneal delivery pathway.

Valve 208 is located along a segment of the circuit leading to the dialyzer 148 and is configured to control inclusion of the dialyzer in the fresh-solution delivery pathway 202. In one configuration, valve 208 is opened so that fresh peritoneal dialysis solution flowing along pathway 202 passes through the dialyzer 148 before reaching the patient connection set, thereby providing an additional filtration barrier between the balancing chamber assembly 128 and the patient. In another configuration, valve 208 is set so that pathway 202 bypasses the dialyzer 148, allowing solution to be delivered directly to the patient connection set when dialyzer filtration is not desired. Valve 208 may also be used during priming or rinsing procedures to direct rinse solution through the dialyzer 148 while maintaining separation from the patient line.

The controller 126 coordinates operation of valves 204, 206, 208, and 210 together with valve sets 134 surrounding the balancing chamber assembly 128 to establish different operating modes for the delivery pathway 202. In a peritoneal dialysis delivery mode, the controller may open valve 210 and configure valve 208 to include the dialyzer 148 or bypass it, while keeping valve 206 closed and valve 204 in an isolated state. In a priming or disinfection mode, the controller may open valves 204 and 206 so that rinse solution from the rinse port is drawn into the upper boundary line and flushed through delivery pathway 202, optionally through the dialyzer 148 under control of valve 208. These combinations of valve states allow the same physical plumbing to be used for therapy delivery, priming, and cleaning while maintaining controlled separation between patient lines and external fluid sources.

FIG. 3 illustrates an example recirculation pathway used to pressurize the spent side of the balancing chamber assembly while remaining hydraulically isolated from the patient. A recirculation loop 302 extends around the outer portion of the hydraulic architecture and returns to a region adjacent the balancing chamber assembly 128. In the configuration of FIG. 3, dialysate or other fluid is driven around loop 302 under control of the controller 126 and a recirculation pump such as the recirculation pump 164 described above. Loop 302 overlays the existing hemodialysis architecture and may be active during peritoneal dialysis operation or during rinsing or disinfection procedures while the patient is disconnected.

The recirculation loop 302 includes an upper horizontal segment routed across the top of the architecture, a left vertical segment extending downward, and a lower horizontal segment returning toward the balancing chamber assembly 128. On the right-hand side of FIG. 3, loop 302 is split into two generally vertical legs that pass alongside the fresh and spent sides of the balancing chambers. These parallel legs of loop 302 allow substantially uniform pressure to be communicated to the spent-side compartments 132 of the balancing chamber assembly 128, thereby moving the internal barriers in a controlled fashion and causing fresh solution to be displaced from the fresh-fluid compartments 130 toward the patient delivery pathway illustrated in FIG. 2. In this manner, loop 302 functions as a closed pressurization path that repurposes the balancing chambers as precision dosing elements without exposing the patient directly to the fluid circulating in loop 302.

Valve 204 is located at an upstream portion of the recirculation loop 302 and controls communication between the loop and an upstream region of the dialysate circuit. When valve 204 is opened in a recirculation mode, dialysate or other fluid from the dialysate side of the machine is permitted to enter loop 302 and circulate around the outer pathway. When valve 204 is closed, the recirculation loop 302 is hydraulically isolated from that upstream region, thereby returning the system to a conventional hemodialysis configuration.

Valve 206 is located along a branch that connects loop 302 to a rinse port. Valve 206 may be opened to draw rinsing or disinfection fluid into the recirculation loop 302 from the rinse port 152 and to distribute that fluid through the balancing chamber assembly 128 and other portions of the circuit during cleaning procedures. Valve 206 may be closed during peritoneal dialysis operation so that loop 302 recirculates only dialysate or other internal fluid without communication to the external rinse connection.

Valve 208 is located along a segment of the dialysate pathway adjacent a dialyzer 148. Valve 208 may be actuated to selectively include or bypass the dialyzer 148 within the recirculation loop 302. In one configuration, valve 208 is opened to permit flow through the dialyzer during disinfection or rinsing procedures so that the dialyzer is flushed by the recirculating fluid. In another configuration, valve 208 is closed so that fluid in loop 302 bypasses the dialyzer and is routed directly toward the balancing chamber assembly 128, thereby concentrating the recirculation-induced pressure on the spent-side compartments 132 without unnecessary flow through the dialyzer.

Valve 310 is positioned along a branch connecting the recirculation loop 302 to the spent side of the balancing chamber assembly 128. When valve 310 is opened, fluid circulating in loop 302 is directed into a pressurization manifold that communicates with the spent-side compartments 132 of the balancing chambers. Pressurization generated by the circulating fluid moves the barriers within the balancing chambers and displaces fresh solution from the fresh-fluid compartments 130 toward the delivery pathway of FIG. 2. When valve 310 is closed, loop 302 is isolated from the balancing chamber assembly 128 such that the chambers revert to conventional balanced-flow operation without recirculation-based pressurization.

The valves 204, 206, 208, and 310 are operated in coordinated fashion by the controller 126 to establish different operating modes. In a peritoneal dialysis delivery mode, the controller may open valves 204 and 310 while closing valve 206 and optionally valve 208, thereby forming a closed recirculation path that pressurizes the spent side of the balancing chamber assembly 128 without including the dialyzer 148 or rinse port. In a disinfection or rinse mode, the controller may open valves 204, 206, and 208 so that chemical disinfectant or rinse solution is drawn in through the rinse port, circulated through the dialyzer 148 and balancing chamber assembly 128, and returned along loop 302. Other combinations of valve states may be used to create additional modes such as partial-loop recirculation, dialyzer-only flushing, or bypass of the balancing chamber assembly 128.

FIG. 3 illustrates an example recirculation pathway 302 configured to apply pressure to a spent-fluid side of the balancing chamber assembly. In the illustrated embodiment, the recirculation pathway 302 forms a closed hydraulic loop and is identified by multiple segments labeled with the same reference numeral to indicate common fluid communication. The recirculation pathway 302 is fluidly coupled to the spent compartments of the balancing chamber such that circulation of fluid within the loop generates a controllable pressure acting on a movable barrier within the chamber.

Valve 204 may be actuated to selectively place the recirculation loop 302 in fluid communication with the main dialysate circuit or to isolate the loop during certain operating states. Valve 206 may provide selective access to a rinse or service port and may be used during priming, rinsing, or disinfection. Valve 208 may be located along an inlet to the spent side of the balancing chamber and may regulate recirculation-based pressurization of the spent chamber compartment. Valve 310 may be configured as a vent valve, bypass valve, or pressure-limiting valve to prevent over-pressurization of the recirculation loop or balancing chamber. In some embodiments, one or more pressure sensors or flow sensors may be disposed along the recirculation pathway 302 to monitor loop pressure and verify proper operation.

During operation, a recirculation pump drives fluid through the loop 302, increasing pressure on the spent side of the balancing chamber and causing the movable barrier to displace. Displacement of the barrier meters an equivalent volume of fresh solution from the fresh side of the balancing chamber to the patient line. In this manner, the balancing chamber functions as a dosing pump while retaining its ability to maintain volume balance during other dialysis operating modes.

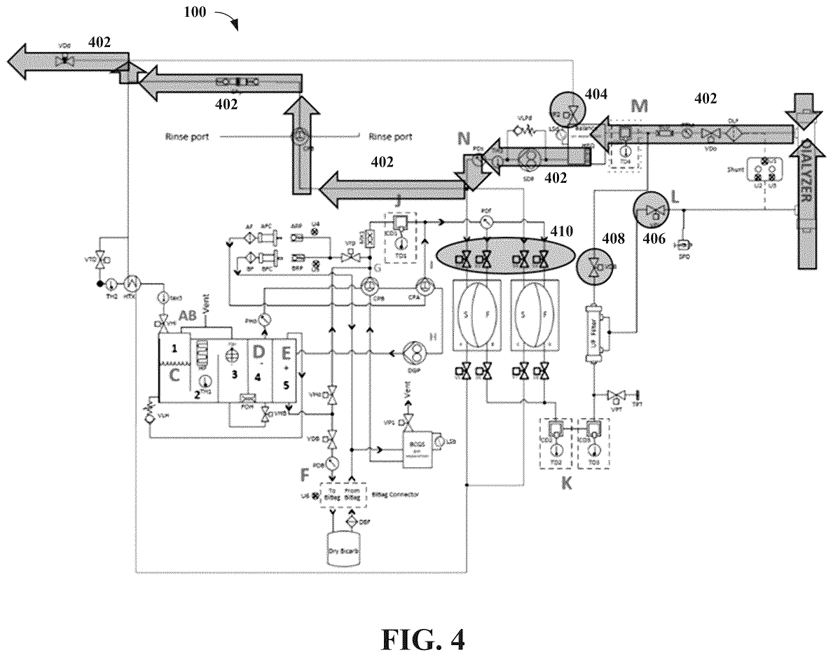

In further embodiments, fluid removal from the patient is accomplished using a pump such as illustrated in FIG. 4. The pump may be configured to provide precise volumetric control over fluid extraction from the peritoneal cavity. In some implementations, a pump used for ultrafiltration during HD therapy is repurposed to perform this function during peritoneal dialysis. This reuse of existing components allows accurate and efficient fluid removal while minimizing system complexity.

Fluid pathway 402 may route treatment fluid through the system. Fluid pathway 402 may include flexible tubing, rigid conduits, molded channels, or cassette-based flow paths and may carry fresh dialysate, prepared solution, rinse solution, or spent effluent depending on operating mode. Fluid pathway 402 may interconnect heating, proportioning, degassing, balancing, and patient-line subsystems and may be configured to tolerate alternating positive and negative pressures associated with balancing-chamber cycling and patient fill and drain operations.

Pump 404 may be positioned along the fluid pathway to advance fluid through the circuit. Pump 404 may, for example, be implemented as a peristaltic pump, diaphragm pump, piston pump, or centrifugal pump suitable for sterile medical fluid handling. Pump 404 may be electronically controlled to establish prescribed flow rates or volumetric delivery profiles and may operate together with downstream valves and balancing structures to achieve accurate therapy delivery. Pump 404 may also be used during priming, rinse-back, or disinfection modes in addition to active treatment.

An additional pump 406 may be located at a different portion of the fluid circuit. The additional pump 406 may, for example, be used to remove spent dialysate or other waste liquid from the system, to return fluid to a drain container, or to establish a desired pressure differential across a balancing chamber or patient interface. Additional pump 406 may operate in coordination with pump 404 to regulate inflow and outflow volumes, thereby supporting ultrafiltration accuracy and maintaining target intraperitoneal pressure levels.

Valve 408 may selectively direct fluid among alternative flow paths in the circuit. Valve 408 may, for example, be implemented as a solenoid-actuated valve, rotary diverter, pinch valve acting on disposable tubing, or cassette-integrated membrane valve actuated pneumatically or mechanically. By actuation of valve 408, the system may direct fluid toward the patient line, a recirculation loop, a drain or waste line, or a rinse or disinfection path. Valve 408 may operate together with pump 404 and additional pump 406 to support alternating fill-and-drain cycles, recirculation modes, solution generation, and priming processes.

Balancing chambers 410 may include two or more compartments separated by a flexible diaphragm or membrane configured to transfer substantially equal fluid volumes between opposing sides of the system. Operation of balancing chambers 410 may match the volume of fluid delivered to the patient with the volume removed from the patient or from the system, thereby enabling precise ultrafiltration control. Balancing chambers 410 may also be associated with sensors configured to detect pressure, membrane displacement, or liquid presence to verify proper cycling and identify potential leak, occlusion, underfill, or overfill conditions. Balancing chambers 410 may additionally participate in priming and rinse operations by alternately filling and emptying selected compartments in response to commanded valve and pump sequences.

The peritoneal dialysis system may utilize many of the components and configurations of an existing HD machine. In some embodiments, the primary changes required for PD functionality are implemented in software that directs valve states and pump operation to establish fluid pathways specific to peritoneal dialysis, as depicted in FIGS. 2-4. Pumps that are conventionally used to deliver acid and bicarbonate concentrate in HD therapy may be reassigned in PD operation to deliver an osmotic agent, electrolytes, and buffer components used in the PD solution chemistry.

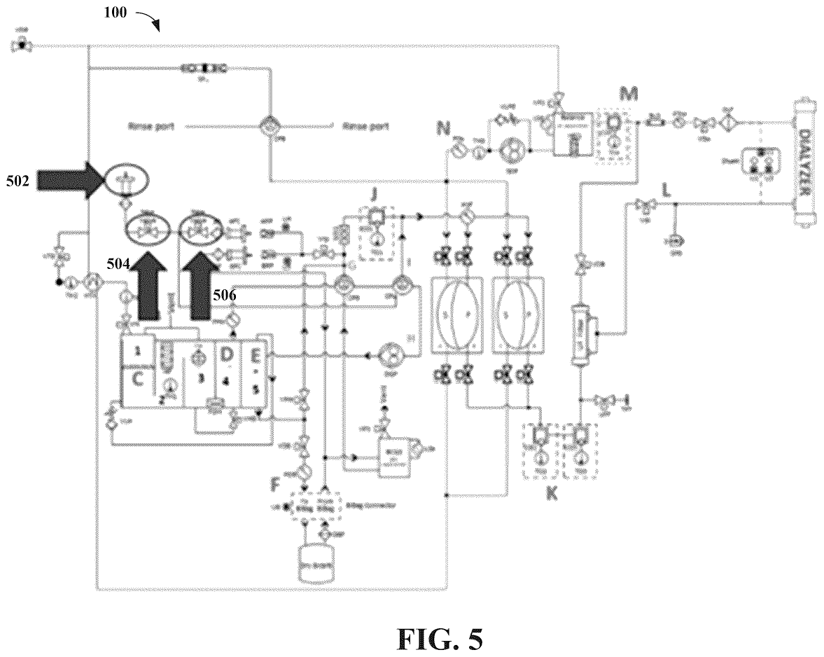

In certain embodiments, a three-stream solution chemistry may be employed without altering the underlying hardware architecture. In other embodiments, a four-stream solution chemistry may be implemented by adapting the HD fluid pathway. FIG. 5 illustrates one such adaptation in which the HD acid line is bifurcated and additional valves are incorporated. This configuration enables the system to alternate delivery between an osmotic agent and electrolytes while still employing the existing HD acid pump as the delivery mechanism.

As shown in FIG. 5, a first arrow 502 (the leftmost horizontal arrow) identifies a portion of an acid delivery line downstream of an acid pump of the hemodialysis architecture and upstream of a bifurcation point. At this location, the acid delivery line is split into two parallel branch lines, each associated with a controllable valve. A second arrow 504 (the left-hand vertical arrow) identifies a valve set along a first branch that, when opened while the valve set identified by arrow 506 is closed, directs flow from the acid pump into a first delivery stream carrying an osmotic agent (for example, a dextrose or icodextrin concentrate). A third arrow 506 (the right-hand vertical arrow) identifies a valve set along a second branch that, when opened while the valve set identified by arrow 504 is closed, directs flow from the acid pump into a second delivery stream carrying electrolytes. Under control of the controller, the valve sets indicated by arrows 504 and 506 are selectively opened and closed to alternately deliver the osmotic-agent stream and the electrolyte stream while continuing to employ the existing acid pump as a shared delivery mechanism. The arrows 502, 504, and 506 schematically illustrate exemplary flow directions and valve locations used to implement the four-stream chemistry configuration of FIG. 5; other valve placements and branching arrangements may be used in alternative embodiments.

The balancing chamber itself provides precision dosing and fluid management capabilities. In certain embodiments, recirculation of spent dialysate generates pressure that allows the balancing chamber to deliver controlled volumes of fresh PD solution to the patient. This architecture allows for accurate ultrafiltration control while enabling fluid transfer previously not achievable using conventional balancing systems.

The systems and methods described herein provide several advantages. First, the balancing chamber is used for precision delivery of PD solution to the patient while retaining its balancing functionality. By pressurizing the spent side through recirculation, the chamber can act as a dosing pump capable of controlled delivery volumes. Second, real-time mixing of PD solution from tap water eliminates the need for prepackaged solutions, thereby reducing storage requirements, transportation costs, and environmental impact. Third, integration of water purification to AAMI standards provides patient safety while allowing use of commonly available tap water supplies, expanding usability in home or resource-limited settings. Fourth, repurposing an HD ultrafiltration pump to remove fluid from the patient allows accurate fluid removal without dedicated new hardware components.

Additional advantages include architectural flexibility to support either three-stream or four-stream chemistries without wholesale redesign of the base HD system. By bifurcating the acid line and adding select valves, the machine can alternate delivery of osmotic agents or electrolytes as needed. Dual ultrafiltration downstream of the mixing point provides ultra-pure PD solution even when mixed in real time, addressing infection and peritonitis risk. In some embodiments, the dialyzer itself may serve as an additional filtration barrier when dialysate is intentionally pushed across the dialyzer membrane, further enhancing the safety of delivered fluid.

FIGS. 2-4 describe efficient fluid pathways for delivering fresh PD solution to the patient, generating pressure on the spent side of the balancing chamber via recirculation, and removing fluid from the patient with a pump. These pathways enable smooth operation and precise fluid volume control. The overall system thus reuses proven HD components and architecture while enabling peritoneal dialysis, convective therapies, and dialysate-to-blood-side fluid transfer functionalities not present in conventional devices.

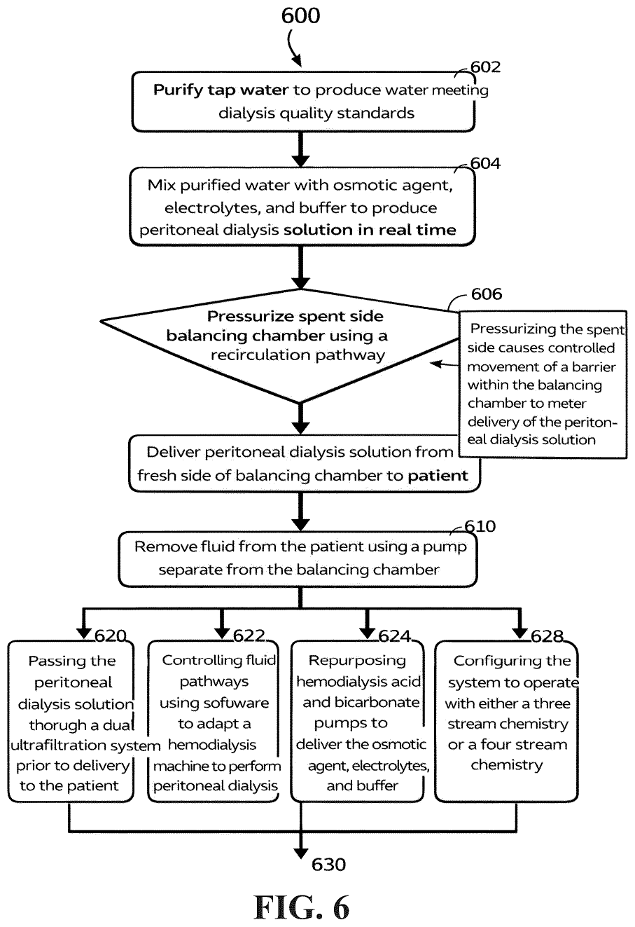

FIG. 6 illustrates an example flow diagram of a method 600 for performing peritoneal dialysis. Method 600 includes a step 602 associated with purifying tap water to produce water meeting dialysis water quality standards, followed by a step 604 associated with mixing the purified water with an osmotic agent, electrolytes, and a buffer to produce peritoneal dialysis solution in real time. A further step 606 is associated with pressurizing a spent side of a balancing chamber using a recirculation pathway. The method additionally includes a step 610 associated with removing fluid from the patient using a pump separate from the balancing chamber. Optional operations illustrated in FIG. 6 include a step 620 associated with passing the peritoneal dialysis solution through a dual ultrafiltration system prior to delivery to the patient, a step 622 associated with controlling fluid pathways using software to adapt a hemodialysis machine to perform peritoneal dialysis, a step 624 associated with repurposing hemodialysis acid and bicarbonate pumps to deliver the osmotic agent, electrolytes, and buffer, and a step 628 associated with configuring the system to operate with either a three-stream chemistry or a four-stream chemistry. An indicator 630 denotes continuation or completion of the depicted method sequence.

Element 600 identifies the overall peritoneal dialysis method shown in FIG. 6. The method 600 reflects execution of the illustrated sequence by a dialysis system operating under automated or semi-automated control. Method 600 may be performed repeatedly in cycles, may be paused and resumed, or may run as part of a larger treatment program including fill, dwell, and drain phases. Method 600 encompasses both mandatory steps and optional operations, and allows the system to provide controlled delivery and removal of peritoneal dialysis fluid.

Element 602 is directed to purifying tap water to produce water meeting dialysis water quality standards. Performing the operation associated with 602 may include receiving municipal tap water and directing it through pretreatment filters, carbon adsorption beds, and particulate filters, regulating the temperature of the water, and removing dissolved gases. The operation may further include subjecting the water to reverse osmosis, ultrafiltration, or deionization processes to remove dissolved solids and contaminants, monitoring conductivity and other quality parameters, flushing lines prior to clinical use, and preventing downstream flow unless measured values are within established dialysis water specifications.

Element 604 is directed to mixing the purified water with an osmotic agent, electrolytes, and a buffer to produce peritoneal dialysis solution in real time. Performing the operation associated with 604 may include proportioning purified water and multiple concentrates using proportioning pumps or metering devices, drawing osmotic agent concentrate such as dextrose or icodextrin from a source container, introducing electrolyte concentrates to establish desired sodium, calcium, magnesium, chloride, and lactate or bicarbonate ion concentrations, and combining the fluids in a mixing manifold. Performing the operation associated with 604 may also include heating or maintaining temperature of the mixed solution, circulating the mixture to promote homogeneity, performing conductivity or temperature verification, and preventing patient delivery unless acceptable composition criteria are satisfied.

Element 606 is directed to pressurizing the spent side of a balancing chamber using a recirculation pathway. Performing the operation associated with 606 may include actuating valves to establish a closed recirculation loop, operating a pump to circulate fluid through that loop, and hydraulically coupling the circulating fluid to the spent side of one or more balancing chambers. Performing the operation associated with 606 may further include generating pressure on the spent side that causes controlled movement of a barrier such as a diaphragm, membrane, or piston within the balancing chamber, thereby metering displacement of fresh peritoneal dialysis solution from the fresh side of the chamber toward the patient pathway. The pressurization may be regulated to set delivery rate, volume, and pressure conditions.

Element 610 is directed to removing fluid from the patient using a pump separate from the balancing chamber. Performing the operation associated with 610 may include operating a dedicated effluent pump to draw spent dialysate and ultrafiltrate from the peritoneal cavity through a patient line, routing the removed fluid toward a drain line or collection container, and monitoring removed volume and pressure to ensure safe extraction. Performing the operation associated with 610 may additionally include modulating pump speed to achieve prescribed ultrafiltration targets, detecting occlusions or disconnections, preventing excessive negative pressure, and coordinating fluid removal with fill and dwell phases of therapy.

Element 620 is directed to passing the peritoneal dialysis solution through a dual ultrafiltration system prior to delivery to the patient. Performing the operation associated with 620 may include routing prepared solution sequentially through two ultrafilter cartridges, removing bacteria, endotoxin, and particulates, providing redundancy for safety, and creating a sterile barrier between upstream system hardware and the patient line. Performing the operation associated with 620 may further include verifying integrity of the ultrafilters, monitoring differential pressure, and diverting solution in response to abnormal filter performance.

Element 622 is directed to controlling fluid pathways using software to adapt a hemodialysis machine to perform peritoneal dialysis. Performing the operation associated with 622 may include executing control logic that configures existing valve arrays and pump drivers into operating states specific to peritoneal dialysis, enabling recirculation-based pressurization of the balancing chamber, modifying alarm thresholds and monitored parameters, and establishing solution preparation and delivery sequences appropriate for peritoneal therapy. Performing the operation associated with 622 may additionally include updating user-interface displays, guiding the operator through peritoneal dialysis setup procedures, and disabling unnecessary hemodialysis pathways.

Element 624 is directed to repurposing hemodialysis acid and bicarbonate pumps to deliver the osmotic agent, electrolytes, and buffer. Performing the operation associated with 624 may include commanding pumps that traditionally meter acid and bicarbonate concentrates in hemodialysis mode to instead meter peritoneal dialysis concentrates, drawing alternative fluids or powders from peritoneal dialysis concentrate containers, and adjusting proportioning ratios to correspond to peritoneal prescription requirements. Performing the operation associated with 624 may further include rerouting flow through alternative branches, controlling valve timing to switch among different concentrate sources, and recalibrating concentration monitoring based on newly assigned functions of the pumps.

Element 628 is directed to configuring the system to operate with either a three-stream chemistry or a four-stream chemistry. Performing the operation associated with 628 may include selecting among solution chemistry modes in software, enabling or disabling concentrate streams, and scheduling delivery of osmotic agent, electrolyte, and buffer streams in desired proportions. Performing the operation associated with 628 may further include implementing a four-stream configuration by bifurcating an acid line, selectively actuating valves to alternate delivery of osmotic agent and electrolytes, and coordinating those alternations with balancing-chamber and proportioning operations to achieve the desired final solution composition.

Element 630 denotes continuation or completion of the method. Performing the operation associated with 630 may include terminating the treatment, repeating some or all of the preceding steps for subsequent cycles, transitioning to additional therapies such as priming of blood tubing, fluid bolus delivery, rinse back, hemofiltration, or hemodiafiltration, or storing treatment data for later review.

The preceding disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like) and may be used interchangeably with “one or more.” The phrase “only one” or similar language is used where only one item is intended. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

One or more elements or aspects or steps, or any portion(s) thereof, from one or more of any of the systems and methods described herein, may be combined with one or more elements or aspects or steps, or any portion(s) thereof, from one or more of any of the other systems and methods described herein and combinations thereof, to form one or more additional implementations and/or claims of the present disclosure.

One or more components, steps, features, and/or functions illustrated in the figures may be rearranged and/or combined into a single component, feature, or function. Additional elements, components, steps, and/or functions may also be added without departing from the disclosure. The apparatus, devices, and/or components illustrated in the Figures may be configured to perform one or more of the methods, features, or steps described in the Figures.

Reference in the specification to “one embodiment” or “an embodiment” means that a particular feature, structure, or characteristic described in connection with the embodiment is included in at least one embodiment of the invention. The appearances of the phrase “in one embodiment” in various places in the specification do not necessarily refer to the same embodiment.

The figures and the description describe certain embodiments by way of illustration only. One skilled in the art will readily recognize from the following description that alternative embodiments of the structures and methods illustrated herein may be employed without departing from the principles described herein. Reference will now be made in detail to several embodiments, examples of which are illustrated in the accompanying figures. It is noted that wherever practicable similar or like reference numbers may be used in the figures to indicate similar or like functionality.

The foregoing description of the embodiments of the present invention has been presented for the purposes of illustration and description. It is not intended to be exhaustive or to limit the present invention to the precise form disclosed. Many modifications and variations are possible in light of the above teaching. It is intended that the scope of the present invention be limited not by this detailed description, but rather by the claims of this Application. As will be understood by those familiar with the art, the present invention may be embodied in other specific forms without departing from the spirit or essential characteristics thereof. Likewise, the naming and division of the mechanisms, components, and features are not mandatory or significant, and the mechanisms that implement the present invention or its features may have different names, divisions and/or formats.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” The word “exemplary” is used herein to mean “serving as an example, instance, or illustration.” Any aspect described herein as “exemplary” is not necessarily to be construed as preferred or advantageous over other aspects. Unless specifically stated otherwise, the term “some” refers to one or more. Combinations such as “at least one of A, B, or C,” “one or more of A, B, or C,” “at least one of A, B, and C,” “one or more of A, B, and C,” and “A, B, C, or any combination thereof” include any combination of A, B, and/or C, and may include multiples of A, multiples of B, or multiples of C. Specifically, combinations such as “at least one of A, B, or C,” “one or more of A, B, or C,” “at least one of A, B, and C,” “one or more of A, B, and C,” and “A, B, C, or any combination thereof” may be A only, B only, C only, A and B, A and C, B and C, or A and B and C, where any such combinations may contain one or more member or members of A, B, or C. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. The words “module,” “mechanism,” “element,” “device,” and the like may not be a substitute for the word “means.” As such, no claim element is to be construed as a means plus function unless the element is expressly recited using the phrase “means for.”

1. A dialysis system comprising:
a water purification module configured to receive tap water and produce purified water meeting dialysis water quality standards;
a mixing subsystem configured to mix the purified water with an osmotic agent, electrolytes, and a buffer to produce a peritoneal dialysis solution in real time;
a balancing chamber having a fresh side and a spent side separated by a movable barrier;
a fluid delivery path configured to deliver the peritoneal dialysis solution from the fresh side of the balancing chamber to a patient;
a recirculation pathway coupled to the spent side of the balancing chamber and configured to pressurize the spent side; and
a controller configured to control fluid flow through the balancing chamber to deliver a controlled volume of the peritoneal dialysis solution to the patient.

2. The dialysis system of claim 1, wherein the balancing chamber is configured to operate as a dosing pump by pressurizing the spent side through recirculation of dialysate.

3. The dialysis system of claim 1, further comprising a dual ultrafiltration system positioned downstream of the mixing subsystem and upstream of the patient, the dual ultrafiltration system configured to remove contaminants from the peritoneal dialysis solution.

4. The dialysis system of claim 1, wherein the system is configured to reuse a hemodialysis fluid architecture to perform peritoneal dialysis by controlling fluid pathways using software.

5. The dialysis system of claim 4, wherein an acid pump and a bicarbonate pump of a hemodialysis machine are repurposed to deliver the osmotic agent, electrolytes, and buffer used to produce the peritoneal dialysis solution.

6. The dialysis system of claim 1, further comprising a fluid removal pump configured to remove fluid from the patient independently of the balancing chamber.

7. The dialysis system of claim 6, wherein the fluid removal pump comprises a pump originally configured for ultrafiltration in a hemodialysis system.

8. The dialysis system of claim 1, wherein the system is configurable to operate using a three stream chemistry without modification of a hemodialysis architecture.

9. The dialysis system of claim 1, wherein the system is configurable to operate using a four stream chemistry by bifurcating an acid delivery line and adding at least two valves to selectively deliver the osmotic agent and electrolytes.

10. The dialysis system of claim 9, wherein an existing acid pump of a hemodialysis architecture is used as a delivery mechanism for both the osmotic agent and the electrolytes.

11. The dialysis system of claim 1, wherein the peritoneal dialysis solution is delivered through a dialyzer, and wherein the dialyzer provides additional filtration prior to delivery to the patient.

12. A method of performing peritoneal dialysis, comprising:
purifying tap water to produce water meeting dialysis water quality standards;
mixing the purified water with an osmotic agent, electrolytes, and a buffer to produce a peritoneal dialysis solution in real time;
pressurizing a spent side of a balancing chamber using a recirculation pathway;
delivering the peritoneal dialysis solution from a fresh side of the balancing chamber to a patient; and
removing fluid from the patient using a pump separate from the balancing chamber.

13. The method of claim 12, wherein pressurizing the spent side causes controlled movement of a barrier within the balancing chamber to meter delivery of the peritoneal dialysis solution.

14. The method of claim 12, further comprising passing the peritoneal dialysis solution through a dual ultrafiltration system prior to delivery to the patient.

15. The method of claim 12, further comprising controlling fluid pathways using software to adapt a hemodialysis machine to perform peritoneal dialysis.

16. The method of claim 15, further comprising repurposing hemodialysis acid and bicarbonate pumps to deliver the osmotic agent, electrolytes, and buffer.

17. The method of claim 12, further comprising configuring the system to operate with either a three stream chemistry or a four stream chemistry.

18. The method of claim 17, wherein the four stream chemistry is achieved by bifurcating an acid line and selectively actuating valves to alternate delivery of the osmotic agent and electrolytes.

19. The method of claim 12, further comprising delivering the peritoneal dialysis solution through a dialyzer to provide additional filtration.

20. The method of claim 12, wherein the method supports additional therapies including priming of blood tubing, fluid bolus delivery, rinse back, hemofiltration, or hemodiafiltration.
